(19) United States
(12) Patent Application Publication
(10) Pub. No.: US 2021/0180138 A1
(43) Pub. Date: Jun. 17, 2021

(54) METHODS FOR PROGNOSIS AND TREATMENT OF SOLID TUMORS

(71) Applicant: CUREWIZE HEALTH LTD., Yokneam Elite (IL)

(72) Inventors: Keren SHICHRUR, Kiryat Ono (IL); Isaac YANIV, Petah Tikva (IL); Smadar AVIGAD, Petah Tikva (IL)

(73) Assignee: Mor Research Applications Ltd., Tel Aviv (IL)

(21) Appl. No.: 16/761,806

(22) PCT Filed: Nov. 6, 2018
(86) PCT No.: PCT/IL2018/051180

§ 371 Date: May 6, 2020

Related U.S. Application Data
(60) Provisional application No. 62/581,786, filed on Nov. 6, 2017.

(51) Int. Cl. C12Q 1/6886

(52) U.S. Cl. CPC C12Q 1/6886; C12Q 2600/178; C12Q 2600/158; C12Q 2600/106

(57) ABSTRACT

Described herein is the correlation between miR-451 expression and the increased benefit of a treatment modality of solid tumors that effects miR-451 modulated metabolic pathways. Such modalities include NAMPT inhibitors and/or PARP inhibitors. Also described herein are methods for treatment of solid tumors that include determining whether treatment of the tumor would benefit from inhibition of NAMPT and/or PARP and if so, treating the patient afflicted with the tumor with NAMPT and/or PARP inhibitors.

### CROSS-REFERENCE TO RELATED APPLICATIONS

This application claims the benefit of U.S. Provisional Patent Application No. 62/581,786 filed on Nov. 6, 2017; the contents of which are incorporated by reference herein in their entirety.

### FIELD

Provided herein are methods for prognosis of a treatment modality and treatment of solid tumors.

### BACKGROUND

MiR-451 was reported to be frequently dysregulated in many types of malignancies in humans including in lung cancer, gastric cancer, breast cancer, glioma and leukemia, indicating that miR-451 might play a critical role in oncogenesis. (See Bian H B, Pan X, Yang J S, Wang Z X, De W). Upregulation of microRNA-451 increases cisplatin sensitivity of non-small cell lung cancer cell line (A549). (J Exp Clin Cancer Res 2011; 30:20 and Pan X, Wang R, Wang Z X. The potential role of miR-451 in cancer diagnosis, prognosis, and therapy. Mol Cancer Ther 2013; 12(7):1153-62). Recent studies addressed the clinical applications of miR-451 as a diagnostic or prognostic biomarker reporting that miR-451 is associated with the clinical outcome of human patients with cancer, such as in lung cancer, hepatocellular cancer, esophageal squamous cancer, and leukemia (Pan, et al.). Interestingly, the mode of action of miR-451 was different is different malignances. In some, miR-451 was down-regulated, and in others was up-regulated. In glioblastoma patients, elevated miR-451 is associated with shorter survival (Godlewski J, Bronisz A, Nowicki M O, Chiocca E A, Lawler S. microRNA-451: A conditional switch controlling glioma cell proliferation and migration. Cell Cycle 2010; 9(14):2742-8), while in gastric and colorectal cancer tissues miR-451 expression was decreased compared with non-tumor tissues (Bandres E, Bitarte N, Arias F, Agorreta J, Fortes P, Agirre X, et al. microRNA-451 regulates macrophage migration inhibitory factor production and proliferation of gastrointestinal cancer cells. Clin Cancer Res 2009; 15(7):2281-90). Also in acute lymphoplastic leukemia (ALL) samples, miR-451 was shown to be down-regulated compared to healthy controls (Bandres, et al.). Taken together, these data support additional evidence that the expression of miR-451 is tissue specific.

Nicotinamide phosphoribosyltransferase (NAMPT) is an enzyme which converts nicotinamide to nicotinamide mononucleotide, enabling Nicontinamide adenine dinucleotide (NAD+) biosynthesis.

Inhibitors of the enzyme NAMPT have been suggested for treatment of various diseases including types of cancer. An exemplary NAMPT inhibitor is the compound known as FK866 (daporinad) which has been tested in various diseases.

Redundancy is a common feature of miRNA targeting. To control the expression of a transcript, miRNAs bind their seed region with an miRNA-responsive motif generally located in the 3′ UTR of the target mRNA. Each miRNA is predicted to repress the expression of thousands of mRNAs, and in turn, each mRNA can be targeted by several hundred different miRNAs (Di Leva G, Garofalo M, Croce C M. MicroRNAs in cancer. Annu Rev Pathol 2014; 9:287-314). Previous studies reported that NAMPT is a direct target of miR-26b in colorectal cancer cells (Zhang C, Tong J, Huang G. Nicotinamide phosphoribosyl transferase (Nampt) is a target of microRNA-26b in colorectal cancer cells. PLoS One 2013; 8(7):e69963), miR-182 in HIV expressing cells (TZM-bl) (Chen X Y, Zhang H S, Wu T C, Sang W W, Ruan Z. Down-regulation of NAMPT expression by miR-182 is involved in Tat-induced HIV-1 long terminal repeat (LTR) transactivation. Int J Biochem Cell Biol 2013; 45(2):292-8) and miR-34a (Choi S E, Fu T, Seok S, Kim D H, Yu E, Lee K W, et al. Elevated microRNA-34a in obesity reduces NAD+ levels and SIRT1 activity by directly targeting NAMPT. Aging Cell 2013; 12(6):1062-72). Thus, each cancer type represents different targets profile of the dysregulated miRs.

NAMPT protein is expressed in certain normal tissues with the highest levels in liver, pancreas, adrenal gland and muscle. Several different human malignant tumors have been demonstrated to over-express NAMPT. Although different human malignant tumors have been demonstrated to over-express NAMPT, the expression level in each malignancy is different.

FK866 (known also as AP0866 or WK175) is a highly specific, noncompetitive NAMPT inhibitor inducing a gradual NAD+ depletion, ATP depletion, and delayed cell death by apoptosis. Administration of FK866 in vivo suppresses growth of several cancerous cell lines. Different cell lines represent different sensitivity levels to FK866 based on LD50 values. While LD50 value of colon cancer cell line (HCT-116) was 9.0±3.2, LD50 value of prostate cancer (PC-3) was half (4.8±3.0) (Olesen U H, Thougaard A V, Jensen P B, Sehested M. A preclinical study on the rescue of normal tissue by nicotinic acid in high-dose treatment with AP0866, a specific nicotinamide phosphoribosyltransferase inhibitor. Mol Cancer Ther 2010; 9(6):1609-17), meaning that prostate cancer was more sensitive to FK866 than colon cancer. Thus, different malignancies present different sensitivities to NAMPT inhibitor.

Several groups have used the NAMPT expression as a biomarker for determining treatment response to NAMPT inhibitors. However, contradictory results have been obtained (Barraud M, Gamier J, Loncle C, Gayet O, Lequeue C, Vasseur S, et al. A pancreatic ductal adenocarcinoma subpopulation is sensitive to FK866, an inhibitor of NAMPT. Oncotarget 2016, and Olesen U H, Hastrup N, Sehested M. Expression patterns of nicotinamide phosphoribosyltransferase and nicotinic acid phosphoribosyltransferase in human malignant lymphomas. APMIS 2011; 119(4-5):296-303). Barraud et al. indicated that increased resistance to FK866 correlated with high expression levels of NAMPT transcript. On the other hand, CLL cells that express higher levels of NAMPT were more sensitive to FK866 (Gehrke I, Bouchard E D, Beiggi S, Poeppl A G, Johnston J B, Gibson S B, et al. On-target effect of FK866, a nicotinamide phosphoribosyl transferase inhibitor, by apoptosis-mediated death in chronic lymphocytic leukemia cells. Clin Cancer Res 2014; 20(18):4861-72). Furthermore, heterogeneous NAMPT protein levels between CLL patients did not predict sensitivity to FK866 at all (Gehrke et al.). Moreover, in multiple myeloma cell lines and primary cells there was no correlation between NAMPT levels and cytotoxic response to FK866 (Cea M, Cagnetta A, Fulciniti M, Tai Y T, Hideshima T, Chauhan D, et al. Targeting NAD+ salvage pathway induces autophagy in multiple myeloma cells via mTORC1 and extracellular signal-regulated kinase (ERK1/2) inhibition. Blood 2012; 120(17):3519-29). NAMPT expression in human tissues is differential and could not serve as a reliable biomarker for patient selection for FK866 treatment. These studies emphasize that different types of malignancies show a different FK866 treatment response, independent from the level of NAMPT expression, and so the expression and function of biomarkers are also tissue specific.

Thus, a continuing need exists for identification of patients that could benefit from treatment modalities that that affect (inhibit or induce) miR-451-related metabolic pathways.

### SUMMARY

The current inventors have shown that NAMPT expression may be downregulated in cells from various malignancies when those cells are transfected to over-express miR-451 mimic.

The current inventors have also shown that a biomarker known as miR-451 present within the body of certain patients having a solid tumor, optionally selected from the group consisting of prostate, colon, and breast cancer, may be used for a prognostic method of a treatment modality, wherein the treatment modality affects one or more metabolic pathways associated with miR-451 expression. In particular, the metabolic pathway associated with miR-451 expression is a NAMPT pathway.

Methods described herein may be useful to determine if using an inhibitor of NAMPT may be particularly effective relative to other patients having different levels of the biomarker.

Additionally provided herein are methods for treatment of solid tumors, in particular, prostate, colon and breast cancer.

Further methods provided herein relate to methods for prognosis of a treatment modality and treatment of a solid cancerous tumor in a patient, comprising: obtaining a biological sample from the patient; determining a level of expression of miR-451 in the biological sample; assessing if a treatment modality that affects one or more metabolic pathways associated with miR-451 expression will result in a positive prognosis for the patient if the level of expression of miR-451 is below a predetermined level; and treating the patient with a treatment modality that affects a metabolic pathway associated with miR-451 expression.

Methods provided herein further relate to methods for treatment of prostate, colon or breast cancer in a patient comprising: obtaining a biological sample from the patient, determining the level of expression of miR-451 in the biological sample, and treating the patient with an inhibitor of the NAMPT pathway if the level of expression of miR-451 in the biological sample is below a predetermined level. The inhibitor of the NAMPT pathway may be administered in the form of a monotherapy or a combination therapy with another agent or therapy.

The foregoing and other objects, features, and advantages will become more apparent from the following detailed description, which proceeds with reference to the accompanying figures.

### BRIEF DESCRIPTION OF THE DRAWINGS

FIG. 1 depicts a bar graph showing NAMPT expression levels in three types of malignant cells which have been transfected to express a miR-451 mimic sequence and a miR-451 scramble sequence;

FIG. 2 depicts a bar graph showing relative cell viability of prostate cancer (PC3) cells, colon cancer cells (CACO2) and breast cancer cells (MCF7) upon administration of various dosages of the NAMPT inhibitor FK866 or a control (DMSO);

FIG. 3 depicts a bar graph showing relative expression levels of miR-451 in prostate cancer (PC3) cells and breast cancer (MCF7) cells transfected with either a miR-451 mimic, antagomiR-451, or a scramble miR sequence;

FIGS. 4A and 4B depict line graphs showing growth of tumors over time in immunodeficient mice injected with transfected cells from the breast cancer MCF7 cell line (4A) and the prostate cancer PC3 cell line (4B) that has been transfected to express antagomiR-451 (A, dotted line), miR-451 (M, solid line) or a scrambled miR-451 control sequence (C, dashed line);

FIG. 5A depicts a line graph showing growth of tumors over time in immunodeficient mice injected with transfected cells from the prostate cancer PC3 cell line which have been transfected with antagomiR-451, said mice being treated with NAMPT inhibitor (PC3 A+, solid line) or untreated (PC3 A, dotted line);

FIG. 5B depicts a line graph showing growth of tumors over time in immunodeficient mice injected with transfected cells from the prostate cancer PC3 cell line which have been transfected with miR-451, said mice being treated with NAMPT inhibitor (PC3 M+, solid line) or untreated (PC3 M, dotted line);

FIG. 5C depicts a line graph showing growth of tumors over time in immunodeficient mice injected with transfected cells from the prostate cancer PC3 cell line which have been transfected with a scramble miR sequence, said mice being treated with NAMPT inhibitor (PC3 C+, solid line) or untreated (PC3 C, dotted line);

FIG. 6 depicts a flow diagram showing a method for treatment of a patient having a malignancy comprising a determination of miR-451 amounts in a biological sample of the patient; and

FIG. 7 depicts a flow diagram showing a method for determining biomarker thresholds for treatment of patients.

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand.

SEQ ID NO: 1 is the nucleotide sequence of miR-451 mimic.

SEQ ID NO: 2 is the scramble nucleotide sequence of miR-451 which was used as a control.

SEQ ID NO: 3 is the nucleotide sequence of antagomiR-451.

### DETAILED DESCRIPTION

### I. Terms

Unless otherwise noted, technical terms are used according to conventional usage, which for example can be found in Benjamin Lewin, Genes V, published by Oxford University Press, 1994 (ISBN 0-19-854287-9); Kendrew et al. (eds.). The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

Abnormal: Deviation from normal characteristics. Normal characteristics can be found in a control, a standard for a population, etc. For instance, where the abnormal condition is a disease condition, such as prostate cancer, a few appropriate sources of normal characteristics might include an individual who is not suffering from the disease, or a population who did not experience a particular prognosis outcome of the disease, such as relapse. Similarly, abnormal may refer to a condition that is associated with a disease or disease relapse. The term “associated with” includes an increased risk of developing the disease or a relapse thereof. For instance, a certain abnormality (such as an abnormality in expression of a miRNA) can be described as being associated with the biological condition of relapse. Controls or standards appropriate for comparison to a sample, for the determination of abnormality, such as in the determination of an expression cutoff value, also described herein as a “predetermined value”, include samples believed to be normal as well as laboratory-determined values, even though such values are possibly arbitrarily set, and keeping in mind that such values may vary from laboratory to laboratory. Laboratory standards and values may be set based on a known or determined population value and may be supplied in the format of a graph or table that permits easy comparison of measured, experimentally determined values.

Adolescent: A human aged between 15 and 19, inclusive.

Adult: A human aged 20 or over.

Altered expression: Expression of a biological molecule (for example, a miRNA) in a subject or biological sample from a subject that deviates from expression if the same biological molecule in a subject or biological sample from a subject having normal or unaltered characteristics for the biological condition associated with the molecule. Normal expression can be found in a control, a “predetermined value” as described herein, a standard for a population, etc. Altered expression of a biological molecule may be associated with a disease or condition thereof, such as relapse.

Amplification: When used in reference to a nucleic acid, any technique that increases the number of copies of a nucleic acid molecule in a sample or specimen. An example of amplification is the polymerase chain reaction (in all of its forms), in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Biological Sample: Any sample that may be obtained directly or indirectly from an organism, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organs, and/or extracts of tissues, cells (such as, fibroblasts, peripheral blood mononuclear cells, or muscle cells), bone marrow, or organs. A sample is collected or obtained using methods well known to those skilled in the art.

cDNA (complementary DNA): A piece of DNA lacking internal, non-coding segments (introns) and transcriptional regulatory sequences. cDNA can also contain untranslated regions (UTRs), such as those that are responsible for translational control in the corresponding RNA molecule. cDNA is synthesized in the laboratory by reverse transcription from RNA extracted from cells.

Chemotherapeutic agent: An agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Examples of chemotherapeutic agents include ICL-inducing agents, such as melphalan (Alkeran™), cyclophosphamide (Cytoxan™), cisplatin (Platino™) and busulfan (Busilvex™, Myleran™). As used herein generally, a chemotherapeutic agent includes biologic agents employed as antineoplastic agents, including antibody and nucleic acid agents (i.e. biological chemotherapeutic agents).

Control: A reference standard. A control can be a known value indicative of basal expression of a diagnostic molecule such as miR-451, sometimes referred to as a “predetermined value”. In particular examples a control sample is taken from a subject that is known not to have a disease or condition. In other examples a control is taken from the subject being diagnosed, but at an earlier time point, either before disease onset or prior to or at an earlier time point in disease treatment. A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%. In a further particular example, the control expression value of an miRNA of interest was set as the upper third quartile or median of a group of patients before starting disease treatment.

Detect: To determine if an agent (such as a signal or particular nucleic acid probe) is present or absent. In some examples, this can further include quantification.

Determining expression of a gene product: Detection of a level of expression (for example a nucleic acid) in either a qualitative or a quantitative manner. In one example, it is the detection of a miRNA, as described herein.

Diagnosis: The process of identifying a disease or a predisposition to developing a disease or condition, for example cancer or its relapse, by its signs, symptoms, and results of various tests and methods, for example the methods disclosed herein. The conclusion reached through that process is also called “a diagnosis.” A subject diagnosed with a disease or condition is understood to be “afflicted” with the disease or condition.

Label: A detectable compound or composition that is conjugated directly or indirectly to another molecule to facilitate detection of that molecule. Specific, non-limiting examples of labels include radioactive isotopes, enzyme substrates, co-factors, ligands, chemiluminescent or fluorescent agents, haptens, and enzymes.

Malignancy: A disease in which abnormal cells divide and can invade nearby tissues.

Mammal: This term includes both human and non-human mammals. Similarly, the term subject includes both human and veterinary subjects.

MicroRNA (miRNA): Short, single-stranded RNA molecule of 18-24 nucleotides long. Endogenously produced in cells from longer precursor molecules of transcribed non-coding DNA, miRNAs can inhibit translation, or can direct cleavage of target mRNAs through complementary or near-complementary hybridization to a target nucleic acid (Boyd, Lab Invest., 88:569-578, 2008). As used herein, a “microRNA sequence” includes both mature miRNA sequences and precursor sequences such as pri-miRNA, pre-miRNA, and the like.

miR-451: a short, single-stranded RNA molecule found in humans, the sequence of which is set forth herein as SEQ ID NO: 1.

Oligonucleotide: A plurality of joined nucleotides, between about 6 and about 300 nucleotides in length. An oligonucleotide analog refers to a subclass of oligonucleotides that contain moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules. Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Predicted to benefit from: Indicates that a subject would likely benefit from a particular treatment.

Probes and primers: Nucleic acid probes and primers can be readily prepared based on the nucleic acid molecules provided in this invention. A probe comprises an isolated nucleic acid attached to a detectable label or reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example, in Sambrook et al. (ed.), Molecular Cloning: A Laboratory Manual, 2^{d} ed., vol. 1-3, Cold Spring Harbor Laboratory Press, Cold Spring Harbor, N.Y., 1989 and Ausubel et al. Short Protocols in Molecular Biology, 4^{th} ed., John Wiley & Sons, Inc., 1999.

Primers are short nucleic acid molecules, preferably DNA oligonucleotides 10 nucleotides or more in length. More preferably, longer DNA oligonucleotides can be about 15, 17, 20, or 23 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the PCR or other nucleic-acid amplification methods known in the art.

PCR primer pairs can be derived from a known sequence, for example, by using computer programs intended for that purpose. One of ordinary skill in the art will appreciate that the specificity of a particular probe or primer increases with its length. Thus, in order to obtain greater specificity, probes and primers can be selected that comprise at least 17, 20, 23, 25, 30, 35, 40, 45, 50 or more consecutive nucleotides of the target sequence being amplified.

Prognosis: A probable outcome or course of disease, or the process for determining a probable outcome or course of disease. In particular embodiments, prognosis is the outcome or course of the given disease in the absence of treatment; in other embodiments, it is the outcome course of the disease following a particular treatment.

Quantitative real time PCR (RT-qPCR): A method for detecting and measuring products generated during each cycle of a PCR, which products are proportionate to the amount of template nucleic acid present prior to the start of PCR. The information obtained, such as an amplification curve, can be used to quantitate the initial amounts of template nucleic acid sequence.

Reverse transcription: Production of DNA from an RNA template, by the enzyme reverse transcriptase. The DNA product of a reverse transcription reaction is known as cDNA.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Methods of alignment of sequences for comparison are well known in the art.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence remains hybridized to a perfectly matched probe or complementary strand. Conditions for nucleic acid hybridization and calculation of stringencies can be found in Sambrook et al. (ed.), Molecular Cloning: A Laboratory Manual, 2^{nd} ed., vol. 1-3, Cold Spring Harbor Laboratory Press, Cold Spring Harbor, N.Y., 1989, and Tijssen Laboratory Techniques in Biochemistry and Molecular Biology—Hybridization with Nucleic Acid Probes Part I, Chapter 2, Elsevier, New York, 1993.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization, though waste times also influence stringency. Calculations regarding hybridization conditions required for attaining particular degrees of stringency are standard. The following is an exemplary set of hybridization conditions:

Very High Stringency (Detects Sequences that Share 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share 80% Identity or Greater)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share Greater than 50% Identity)

Hybridization: 6×SSC at RT to 55° C. for 16-20 hours

Wash at least twice: 2×-3×SSC at RT to 55° C. for 20-30 minutes each.

Tumor: An abnormal mass of tissue resulting from improper cell division.

### II. Overview of Several Embodiments

Described herein is a method for prognosis of a treatment modality in a patient afflicted with a solid cancerous tumor, the method comprising: obtaining a biological sample from the patient; determining a level of expression of miR-451 in the biological sample; and if the level of expression of miR-451 is below a predetermined level, correlating the level of expression of miR-451 with a positive prognosis of a treatment modality that affects one or more metabolic pathways associated with miR-451 expression.

In a particular embodiment, the metabolic pathway associated with miR-451 expression is a NAMPT pathway. In an additional embodiment the treatment modality that affects one or more metabolic pathway associated with miR-451 expression is a combination therapy.

In some embodiments the combination therapy includes inhibition of a NAMPT pathway combined with one or more of: inhibition of a PARP pathway, radiation, enhancement of DNA/RNA alkylation, inhibition of thymidylate synthase, administration of a nucleotide antimetabolite, inducing TNF-related apoptosis, inhibition of histone deacetylase, administration of an anti-CD20 antibody, inhibition of a proteasome, or inhibition of lactate dehydrogenase A.

In some embodiments the solid tumor could be prostate cancer, breast cancer, and colon cancer.

Further described herein is a composition for use in treatment of a solid tumor in a patient in need thereof, which includes a therapeutically effective amount of an inhibitor of a NAMPT pathway, if the level of expression of miR-451 in the patient is below a predetermined level.

In particular embodiments of the composition for use above, the inhibition of a NAMPT pathway can be combined with one or more of: inhibition of a PARP pathway, radiation, a DNA/RNA alkylation induction, thymidylate synthase inhibition, administration of a nucleotide antimetabolite, TNF-related apoptosis induction, histone deacetylase inhibition, administration of an anti-CD20 antibody, proteasome inhibition, or a lactate dehydrogenase A inhibition.

In some embodiments of the composition for use above, the level of expression of miR-451 in the patient is determined from a biological sample derived from a tumor from the patient.

In some embodiments of the composition for use above, the inhibitor of the NAMPT pathway is FK866.

In particular embodiments of the composition for use above, the solid tumor is selected from prostate cancer, breast cancer, or colon cancer.

Further described herein is a method for treatment of a solid cancerous tumor in a patient, which includes: detecting a level of expression of miR-451 in a biological sample from the patient; assessing if a treatment modality that affects one or more metabolic pathways associated with miR-451 expression will result in a positive prognosis for the patient, and treating the patient with a treatment modality that affects a metabolic pathway associated with miR-451 expression.

In particular embodiments of the method above, the treatment modality inhibits the metabolic pathway associated with miR-451 expression. Further embodiments of the above method above is the treatment modality induces the metabolic pathway associated with miR-451 expression.

### III. miR-451-Related Methods of Prognosis and Treatment

Described herein are methods for prognosing a patient with a malignancy, such as a solid cancerous tumor. The described methods include detecting the level of miR-451 in a sample from a patient (e.g. detecting the amount of miR-451 in the sample, such as by nucleic acid hybridization), and correlating the expression level detected with a particular prognosis depending on a treatment modality provided to the patient. Once prognosed, the patient can then be provided with a treatment modality that will result in the predicted favorable treatment response.

The generally-described methods are provided in detail by way of reference to the figures.

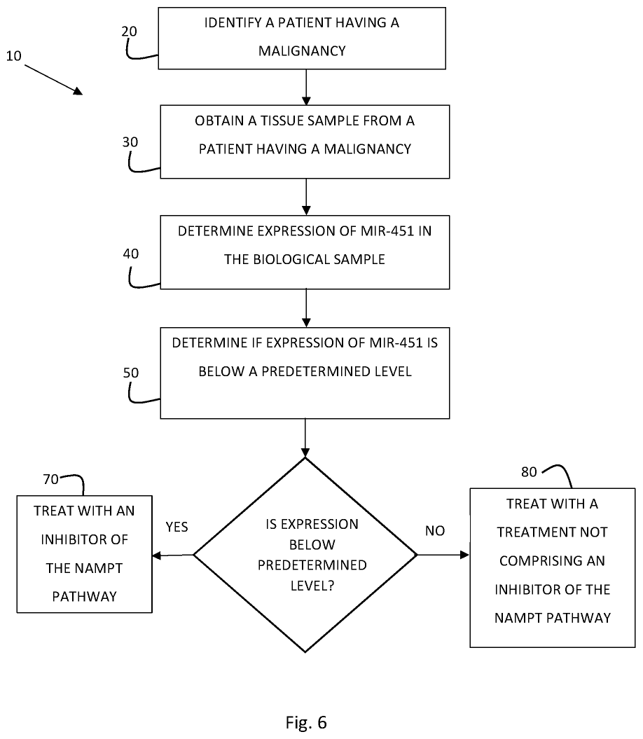

Reference is now made to FIG. 6 which depicts a method 10 for treatment and/or prognosis of a treatment modality, of a patient having a malignancy. Method 10 comprises block 200, comprising identifying a patient having a malignancy. Preferably, the malignancy is prostate cancer, breast cancer or colon cancer.

Method 10 further comprises block 30, comprising obtaining a biological sample from a patient having a malignancy. The biological sample may be from a tumor. The biological sample may be from blood of a patient. The biological sample may be from tissue adjacent to a tumor. The biological sample may be from liquid biopsies. The biological sample for prostate cancer may be from urine. The biological sample for colon cancer may be stool. It will be appreciated that in particular embodiments, the methods do not require either identification of a patient or isolation of the sample, but rather can be initiated with the detection of miR-451 expression as described in block 40.

Method 10 further comprises block 40, comprising determining expression of miR-451 in the biological sample. The miRNA may be detected by any methods known to the art of detecting the presence and levels of a nucleic acid in a sample, including use of standard oligonucleotides primers and probes, each of which can specifically hybridize to a nucleic acid sequence of at least one of miR-451 (SEQ ID NO: 1). Such sequences include sequences that are 100% identical to the reverse complement of SEQ ID NO 1. It is understood that such primers and probes can also be less than identical to the reverse complement of SEQ ID NO 1, such as 98%, 95%, 90%, 85% or even less, and that the design of such primers is well known in the art.

It will be appreciated however, that although certain techniques can utilize standard primers and probes to detect the miRNA level in a sample, the miRNA length (18-24 nt) precludes use of simple amplification techniques. In particular embodiments, miRNA is detected using a DNA microarray, wherein miRNA is extracted from a sample, reverse transcribed, labeled and exposed to DNA microarray with match oligos. miRNA amounts are quantified by measured fluorescence after washing non-specifically bound reverse transcribed sequences. Other methods for RNA quantification of microRNAs may include: RNA-Seq analysis, nanostring technologies, microarray and Real Time qPCR.

In another embodiment, miRNA can be measured by adding a poly-A tract to extracted RNA, reverse transcribing the poly-adenylated RNAs using a poly-A primer, followed by miRNA-sequence specific qPCR, with specific (miRNA-specific) and non-specific (poly-AA) primers.

In yet another embodiment, extracted miRNA is reverse transcribed using an miRNA structure-specific stem-loop primer. The reverse transcribed miRNA sequences are then amplified and quantified by RT-qPCR with miRNA sequence-specific forward primers and a backward primer specific to the miRNA loop. Design of miRNA stem-loop primers and their use in RT-qPCR is described in Kramer, Curr. Prot in Molec. Biol. 15:10, July 2011 (available online at ncbi.nlm.nih.gov/pmc/articles/PMC3152947/). Non-limiting examples of stem-loop primers for use in reverse transcribing miR-451 are based on the description in Kramer (Curr. Prot in Molec. Biol. 15:10, July 2011).

Non-limiting examples of standard nucleic acid detection methods include PCR (in all of its forms, including qPCR), nucleic acid microarrays, Northern blot analysis, and various forms of primer extension.

Primers and probes for use in detecting the described miRNAs can be RNA or DNA, or analogs thereof. Examples of DNA/RNA analogs include, but are not limited to, 2-′O-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. In particular embodiments, the oligonucleotides and analogs can be used alone; in other embodiments, they can be used in combination with one or more additional oligonucleotides or analogs.

In a particular embodiment, the described oligonucleotides are primers or nucleotide probe, for use in detecting the level of expression of miR-451, using a nucleic acid amplification assay including but not limited to Real-Time PCR, micro arrays, PCR, in situ Hybridization and Comparative Genomic Hybridization. Methods and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

In particular embodiments, in addition to detection of the miR of interest, the particular detection methods also utilize primers and/or probes to detect the expression of a nucleic acid to be used as an internal normalizing control. According to this embodiment, the detecting nucleic acid molecules used by the described methods include isolated oligonucleotides that specifically hybridize to a nucleic acid sequence of miR-451; and isolated oligonucleotides that specifically hybridize to a nucleic acid sequence of at least one reference RNA. Non-limiting examples of such reference RNAs include a reference miRNA (whose expression is known to be the same, regardless of disease condition), the 5S ribosomal RNA (rRNA), the U6 small nuclear RNA, or the miRXplore Universal Reference (UR) (Miltenyi biotech), which represents a pool of 979 synthetic miRNA for comparison of multiple samples.

In case of a patient suffering from prostate cancer, a sample used for detection of miRNA may include prostate cancer cells (e.g. from a tumor biopsy or isolated from a sample). In case of a patient suffering from breast cancer, sample used for detection of miRNA may include breast cancer cells. In case of a patient suffering from colon cancer, sample used for detection of miRNA may include colon cancer cells.

In particular embodiments, the methods of determining expression of miR-451 in a biological sample described herein are employed at a single time point after a subject (patient) is diagnosed. In other embodiments, the methods described herein can be used to monitor the progress of a patient and whether their treatment regimen should change over time. Multiple time points can be used in such monitoring, for example, 1, 2, 3, 4, 5, or 6 months or more after diagnosis and treatment initiation (and any time point in between) can be suitable time points to measure the expression of miR-451 in a sample from the patient.

In particular embodiments, the miR-451 expression in the patient is compared to a control, wherein a statistically significant decrease in the miR-451 expression indicates that treatment with a miR-451-related metabolic modulator, such as a NAMPT inhibitor as described herein, would be of benefit. In other embodiments, the miR-451 expression is compared to a predetermined level of miR-451 for similar purposes.

In particular embodiments, the miR-451 expression in the patient is compared to a control, wherein a statistically significant decrease in the miR-451 expression indicates that treatment with a miR-451-related metabolic modulator such as a NAMPT inhibitor, combined with a PARP inhibitor, as described herein, would be of benefit. In other embodiments, the miR-451 expression is compared to a predetermined level of miR-451 for similar purposes.

Method 10 further comprises block 50 comprising determining if expression of miR-451 is below a predetermined level. The predetermined level of miR-451 may be a level, when present in a cell such as a tumor cell of a patient, above which treatment with a treatment modality that affects one or more metabolic pathways associated with miR-451 expression is found to have little or no effect on the malignancy. The predetermined level of miR-451 may be a level, when present in a cell such as a tumor cell of a patient, above which treatment with a NAMPT inhibitor is found to have little or no effect on the malignancy. The predetermined level may be determined based on method 100 for determining a threshold, to be described below.

Method 10 further comprises block 70 comprising treating the patient with a treatment modality that affects a metabolic pathway associated with miR-451 expression, if the expression of miR-451 is below the predetermined level. In a particular embodiment, the treatment modality can be an inhibitor of the NAMPT pathway.

The inhibitor of the NAMPT pathway may be administered in any effective amount sufficient to treat the malignancy.

The inhibitor of the NAMPT pathway may be FK866. FK866 may be administered as 0.126 mg/m²/hr for 4 consecutive days (96 hours), every 3 weeks for a total of 3 cycles.

Other exemplary inhibitors of the NAMPT pathway that can be used in the described methods are listed in Table 1 below:

TABLE 1

| NAMPT | | Available from/ |
|---|---|---|
| inhibitor | Chemical structure | synthesis: |
| FK866 | 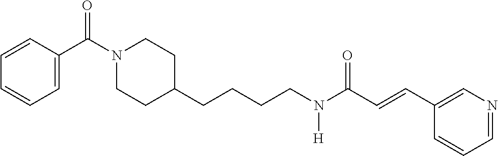 [SMILES: O=C(/C=C/c1cccnc1)NCCCCC1CCN(C(=O)c2ccccc2)CC1] | Sigma-Aldrich. St. Louis, Missouri, USA. |
| GNE-618 | 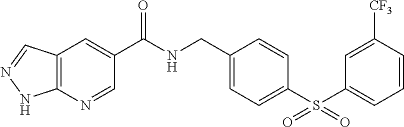 [SMILES: O=C(NCc1ccc(S(=O)(=O)c2cccc(C(F)(F)F)c2)cc1)c1cnc2[nH]ncc2c1] | Zheng et al. Bioorg Med Chem Lett. 2013 Oct. 15;23(20):5488- 97 |
| STF- 118804 | 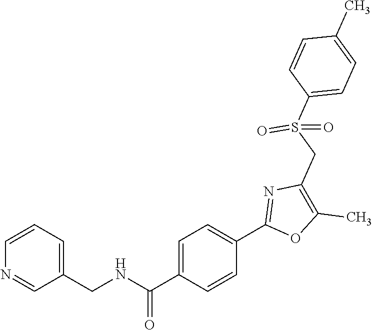 [SMILES: Cc1ccc(S(=O)(=O)Cc2nc(-c3ccc(C(=O)NCc4cccnc4)cc3)oc2C)cc1] | Selleckchem. Houston, Texas. USA |
| GMX1778 | 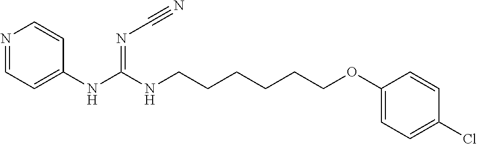 [SMILES: N#C/N=C(\NCCCCCCOc1ccc(Cl)cc1)Nc1ccncc1] | Sigma-Aldrich. St. Louis, Missouri, USA |
| KPT-9274 | 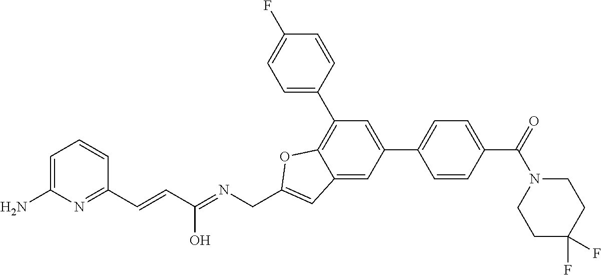 [SMILES: Nc1cccc(/C=C/C(O)=N/Cc2cc3cc(-c4ccc(C(=O)N5CCC(F)(F)CC5)cc4)cc(-c4ccc(F)cc4)c3o2)n1] | MedChemExpress USA. Monmouth Junction, NJ, USA. |

An inhibitor of the NAMPT pathway may be administered as a monotherapy, or in combination with another agent or treatment modality. The other treatment modality may be radiation therapy, administered by methods known to the art and suitable for treatment of the particular malignancy. The other agent may be an anti-cancer pharmaceutical agent (i.e. a chemotherapeutic agent). The anti-cancer agent may be one or more than one of: A DNA/RNA alkylation agent, for example, Temozolomide or Melphalan; a thymidylate synthase inhibitor, for example, 5-fluorouracil or pemetrexed; a nucleotide antimetabolite such as Fludarabine; TNF-related apoptosis-inducing ligand (TRAIL); a histone deacetylase inhibitor such as vorinostat; an anti-CD20 antibody such as rituximab; a proteasome inhibitor such as bortezomib; and a lactate dehydrogenase A inhibitor, such as FX-11.

The other agent may be a PARP inhibitor.

The poly (ADP-ribose) polymerase proteins, otherwise known as PARP, is a family of proteins associated with repair to single-strand breaks in DNA. PARP proteins act through binding to DNA in the vicinity of a single-strand break and initiating signaling to DNA-repair enzymes. One of the members of the PARP family is known as PARP-1.

PARP-1 activity is very high in malignant cells, with a roughly 45-fold higher activity than is seen in normal human lymphocytes, while the PARP-1 protein levels are roughly 23-fold higher. This PARP-1 activity appears to be important in cancer cell survival. Various inhibitors of PARP-1 have been shown to cause cell death in cancer cells. It is suggested that the mechanism of action is that through inhibition of PARP-1, multiple double-strand breaks occur in the DNA of the cancer cell, leading to the death of the cancer cell.

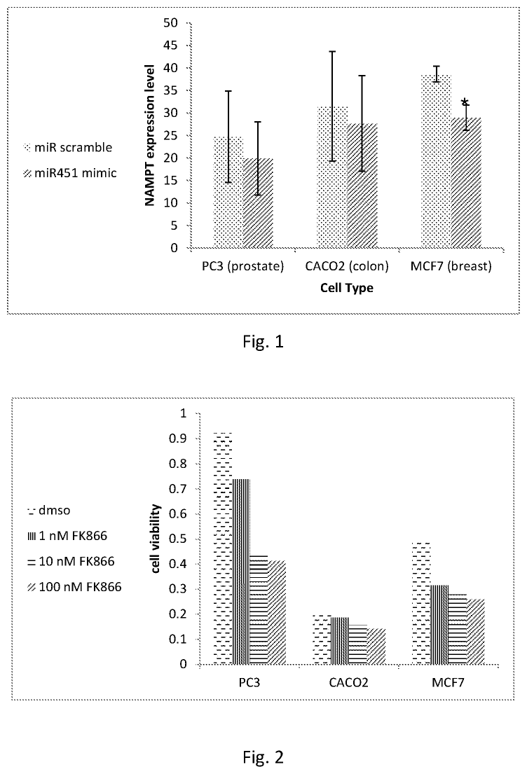

PARP-1 activity is dependent upon Nicotinamide Adenine Dinucleotide (NAD) as a substrate. In cells in which miR-451 is downregulated, it has been shown that levels of NAMPT are increased. (See FIG. 1 for example.) Increased levels of NAMPT leads to higher levels of NAD, which may in turn be used for PARP-1 activity. Since, as mentioned before, PARP activity is high in malignant cells, a PARP inhibitor may be used in such cells to limit PARP activity. On the other hand, in cells in which miR-451 is upregulated, levels of NAMPT are decreased, leading to lower levels of available NAD to act as a substrate for PARP, thereby decreasing PARP activity.

The PARP inhibitor may be an inhibitor selected from one or more than one of the inhibitors in Table 2.

TABLE 2

| PARP | | |
|---|---|---|
| inhibitor | Chemical structure | Available from: |
| Olaparib | 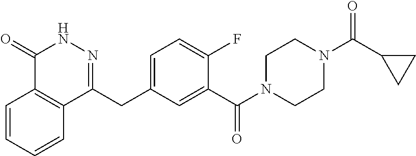 [SMILES: O=C(c1cc(Cc2n[nH]c(=O)c3ccccc23)ccc1F)N1CCN(C(=O)C2CC2)CC1] | AstraZeneca, Cambridge, United Kingdom as Lynparza |
| Rucaparib | 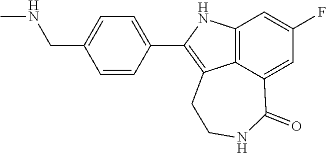 [SMILES: CNCc1ccc(-c2[nH]c3cc(F)cc4c3c2CCNC4=O)cc1] | Clovis Oncology, Boulder, CO, USA as Rubraca |
| Niraparib | 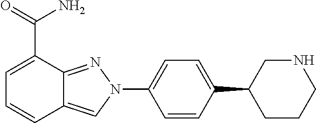 [SMILES: NC(=O)c1cccc2cn(-c3ccc([C@@H]4CCCNC4)cc3)nc12] | Tesaro, Waltham, Massachusetts. USA, as Zejula |
| Veliparib | 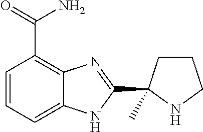 [SMILES: C[C@]1(c2nc3c(C(N)=O)cccc3[nH]2)CCCN1] | Selleckchem. Houston, Texas. USA |
| Talazoparib | 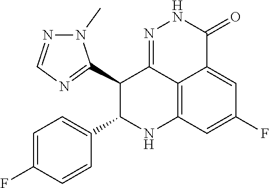 [SMILES: Cn1ncnc1[C@H]1c2n[nH]c(=O)c3cc(F)cc(c23)N[C@@H]1c1ccc(F)cc1] | Selleckchem. Houston, Texas. USA |
| E7449 | 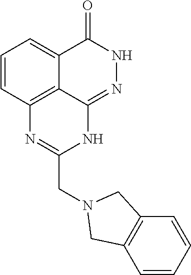 [SMILES: O=c1[nH]nc2c3c(cccc13)N=C(CN1Cc3ccccc3C1)N2] | Selleckchem. Houston, Texas. USA |
| E7016 | 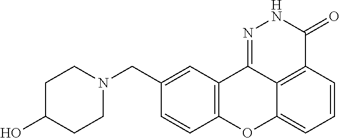 [SMILES: O=c1[nH]nc2c3c(cccc13)Oc1ccc(CN3CCC(O)CC3)cc1-2] | MedKoo Biosciences, Inc. Morrisville, NC, USA. |
| BGB-290 | 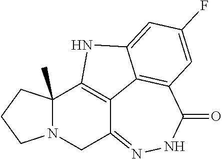 [SMILES: C[C@]12CCCN1CC1=NNC(=O)c3cc(F)cc4[nH]c2c1c34] | MedKoo Biosciences, Inc. Morrisville, NC, USA. |

Method 10 further comprises block 80 comprising treating with an alternate treatment modality other than one that affects a metabolic pathway associated with miR-451 expression, if expression is determined not to be below a predetermined level. Optionally, the alternate treatment may be a treatment not comprising an inhibitor of the NAMPT pathway. The alternate treatment may comprise a chemotherapeutic treatment not comprising an inhibitor of the NAMPT pathway. The alternate treatment may comprise surgery. The alternate treatment may comprise radiotherapy.

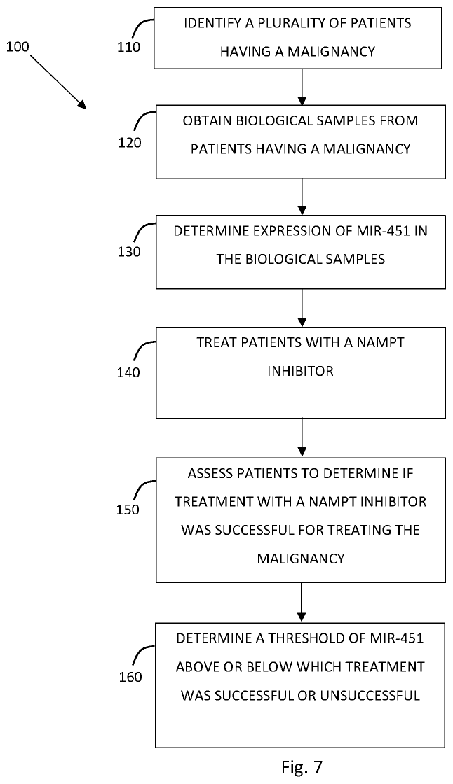

Reference is now made to FIG. 7 which describes method 100 for determination of a threshold level of miR-451 expression. The threshold level may be a level below which a treatment of a patient having a malignancy may be effective with a NAMPT inhibitor.

Method 100 comprises block 110 comprising identifying a plurality of patients having a malignancy. The malignancy may be breast cancer, colon cancer, or prostate cancer.

Method 100 further comprises block 120 comprising obtaining biological samples from patients having a malignancy. Biological samples may be those described with reference to block 30 in FIG. 6.

Method 100 further comprises block 130 comprising determining expression of miR-451 in the biological samples. Determination of expression of miR-451 in the biological samples may be performed as described with reference to block 40 in FIG. 6.

Method 100 further comprises block 140 comprising treating patients with a NAMPT inhibitor. The NAMPT inhibitor may be FK866 or another NAMPT inhibitor described herein.

Method 100 further comprises block 150 comprising assessing patients to determine if treatment with a NAMPT inhibitor was successful for treating the malignancy. Successful treatment may be determined by an endpoint of a trial. For example, successful treatment may be selected from the group consisting of: increased survival time, decrease in tumor size, slowing of growth of tumor size.

Method 100 further comprises block 160 comprising determining a threshold of miR-451 expression below which treatment was successful, or above which, treatment was unsuccessful.

The threshold may be determined by plotting efficacy of treatment versus expression levels of miR-451 in tumor tissue. The plot may show an inverse correlation between expression levels of miR-451 and efficacy of NAMPT inhibitor treatment.

In a population of patients having a malignancy, whose tissue is analyzed for expression of miR-451, a distribution of miR-451 levels of expression may be found. The threshold may be correlated to a percentile of miR-451 levels, below which NAMPT inhibitor is relatively effective and above which NAMPT inhibitor is relatively ineffective. The percentile of miR-451 expression level in the population which may represent the threshold value may be, the 10^{th} percentile, the 15^{th} percentile, the 20^{th} percentile, the 25^{th} percentile, the 30^{th} percentile, the 35^{th} percentile, the 40^{th} percentile, the 45^{th} percentile, or the 50^{th} percentile.

In some embodiments the described methods for prognosis, compositions for use, and methods for treatment, a biological sample from the patient is derived from a tumor of the patient, such as from a biopsy, or are cells detected in a sample from the patient such as blood, lymph, or stool.

In a particular embodiments of the above, the patient is an adult, adolescent, child, or infant.

In some embodiments the compositions for use in treatment of a solid tumor in a patient and the methods employing such compositions include combinations of therapeutically effective agents and/or treatment methods (i.e. treatment modalities). Particular combinations include one or more inhibitors of a NAMPT pathway as described herein and an inhibitor of a PARP pathway as described herein. Other agents that can be combined in treatments or in compositions with NAMPT inhibitors include standard chemotherapeutic or biological anticancer treatment, and non-pharmaceutical treatment modalities including radiation therapy.

In particular embodiments of the method of treatment above, the treatment modalities are combinations with one or more of: radiation, a DNA/RNA alkylation agent, a thymidylate synthase inhibitor, a nucleotide antimetabolite, TNF-related apoptosis-inducing ligand (TRAIL), a histone deacetylase inhibitor, an anti-CD20 antibody, a proteasome inhibitor, or a lactate dehydrogenase A inhibitor. Optionally, the treatment modality that affects a metabolic pathway associated with miR-451 expression is combination therapy.

In particular embodiments of the method for treatment above, the inhibitor of the NAMPT pathway can be FK866.

In particular embodiments of the method for treatment above, the solid tumor can be prostate cancer, breast cancer, and colon cancer.

The following examples are provided to illustrate certain particular features and/or embodiments. These examples should not be construed to limit the disclosure to the particular features or embodiments described.

### EXAMPLES

### Example 1: Sensitivity of Tumor Cell Lines to NAMPT Inhibitor

An in vitro study was performed to determine if miR-451 could act as a biomarker identifying patients that could benefit from treatment with NAMPT inhibitors in adult malignancies, as was previously shown in connection to Acute Lymphoblastic Leukemia (See PCT Application Publication WO 2016/016897). The malignancies studied included: colon cancer, breast cancer and prostate cancer.

A study was performed to determine if NAMPT is regulated by miR-451 in a representative cell line of each malignancy. Cells comprising prostate cancer (PC-3), colon cancer (Caco-2) and breast cancer (MCF7) cell lines were transiently transfected with a miR-451 mimic while “scrambled” miR-451 served as a control. The prostate cancer (PC-3), colon cancer (Caco-2), and breast cancer (MCF7) cells were obtained from ATCC and cultured according to ATCC growth recommendation. Two hundred pmol miR-451 mimic and 200 pmol scramble miR (IDT, Jerusalem, Israel) were transiently transfected by JETPRIME (Polyplus-transfection) into cells according to manufacturer's instructions.

Cells expressing miR-451 mimic showed an increase of 3-fold in the expression levels of the miR compared to the control cells transfected with scramble miR. NAMPT protein expression was determined using FACS as follows:

One to two million transfected cells were collected 48 hours after transfection and fixed using 500 μl BD phosflow fix buffer I (BD Biosciences, San Jose, Calif., USA) for 15 minutes. Then, pellets were washed twice using PBS and resuspended in 500 μl methanol. Samples were incubated for 2 hours in −20° C. For staining, pellets were washed using PBS and resuspended in 10 ml BD cell wash buffer (BD Biosciences, San Jose, Calif., USA) containing 2% FCS. The cells were stained for NAMPT using anti-NAMPT Ab (1:1000) (R&D Systems, MN, USA). The secondary antibody was goat anti-sheep (1:10,000) (R&D Systems, MN, USA). The cells were analyzed on flow cytometer (FACSCalibur, Becton Dickinson, Le Pont-De-Claix, France) using BD CellQuest™ Pro software.

FIG. 1 presents NAMPT protein expression level analyzed by FACS using a specific anti-NAMPT antibody in prostate cancer (PC-3), colon cancer (Caco-2) and breast cancer (MCF7) cell lines. In all cell lines, high levels of miR-451 (mimic) demonstrate a reduction in NAMPT expression compared to control scramble-miR cells. This example shows that reduction of miR-451 levels in a sample increases NAMPT protein levels in prostate, colon, and breast cancer cells.

### Example 2: Cell Viability after Treatment with Various Concentrations of NAMPT Inhibitor

FK866 is a potent NAMPT inhibitor that causes the depletion of intracellular NAD+ levels in the cell and ultimately induces apoptosis and cell death. To characterize the effect of FK866 on prostate cancer (PC-3), colon cancer (Caco-2) and breast cancer (MCF7) cell lines, the effects of FK866 on cell death were evaluated. Cells were either control cells, treated with DMSO only, or were treated for 24 hours with 1 nanomolar (nM), 10 nM and 100 nM of FK866, and cell viability was measured using an XTT tetrazolium colorimetric assay. Following FK866 treatment, a gradual decrease in cell viability was detected (FIG. 2). The results indicate that prostate cancer (PC-3), colon cancer (Caco-2) and breast cancer (MCF7) cell lines are sensitive to NAMPT inhibitor (FK866).

### Example 3: Regulation of miR-451 Expression in Tumor Cells Expressing miR-451 Mimic, AntagomiR and Scramble

The effect of miR-451 expression level on prostate cancer (PC-3), and breast cancer (MCF7) cell line tumor cells was evaluated in-vitro. Stable lines of each malignancy expressing miR-451 mimic and antagomiR-451 were obtained and transfected as in example 1. miR-451 expression levels in each cell line were validated by quantitative Real Time PCR (RT-qPCR). Total RNA was isolated from 10⁷ cells using Qiagen RNeasy isolation kit according to the manufacturer's instruction (Qiagen, Hilden, Germany). 100 ng RNA was converted to cDNA using universal cDNA synthesis kit (Exiqon, Vedbaek, Denmark). RT-qPCR for the miR-451 was performed using Locked-nucleic Acid (LNA™) primers sets (Exiqon, Vedbaek, Denmark). 5S Ribosomal RNA was used as a reference gene. The RT-qPCR reactions were performed in duplicates on the LightCycler 480 (Roche, Rotkreuz, Switzerland) apparatus. The results were expressed as relative expression using the delta Ct method.

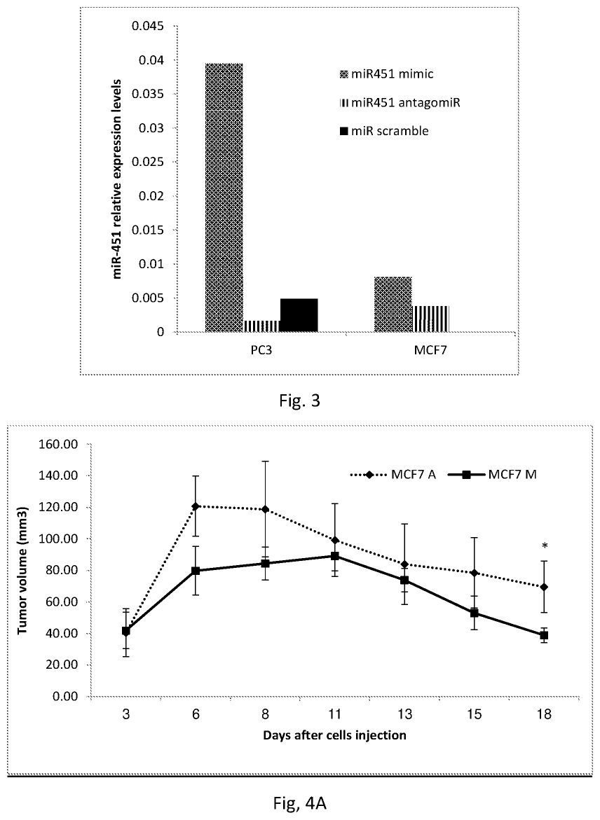

The results show a level in miR-451 twice as high in MCF7 cells transfected with miR-451 mimic as that of MCF7 cells transfected with antagomiR-451 (FIG. 3). PC3 cells expressing miR-451 mimic showed a tenfold increase in miR-451 expression compared with control (PC3 cells transfected with miR scramble). PC3 transfected with antagomiR-451 showed 67% decrease in miR-451 expression compared to PC3 control cells (FIG. 3).

### Example 4A: In Vivo Model Showing Tumor Growth Based on miR-451 Expression

The effect of miR-451 expression levels on tumor cell growth was evaluated in an in-vivo xenograft mice model using MCF7 cells and PC3 cells. Ten million cells harboring antagomiR-451 and miR-451 mimic were injected subcutaneously into the right flanks of 6-8 weeks-old immunodeficient NSG (NOD scid gamma) mice. Ten mice were injected with the MCF7 antagomiR-451 cell line and 11 mice were injected with the miR-451 mimic line. Ten mice were injected with the PC3 antagomiR-451 cell line and the PC3 miR-451 mimic cell line and 8 with the scrambled miR control cell line. The volume of the tumors was measured daily from the day the tumors were clinically evident in all mice.

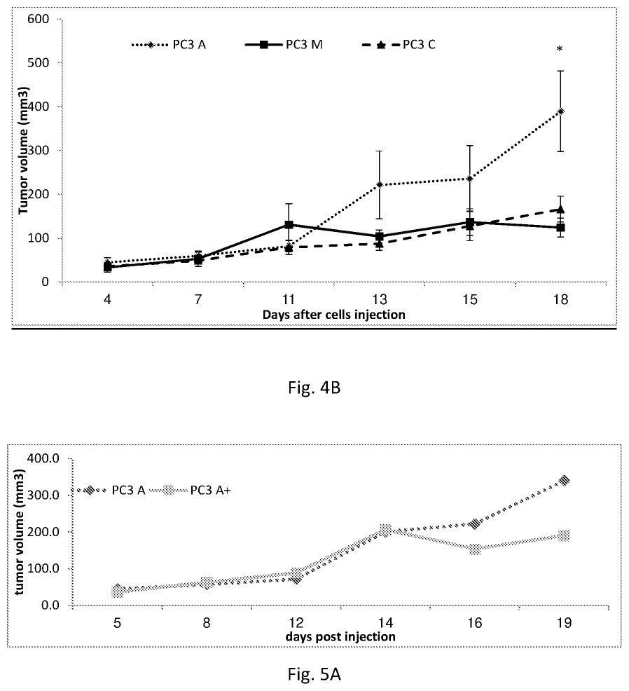

Tumor volume over time is presented in breast and prostate cells lines (FIG. 4). A significant increase in tumor volume was evident at the end of the experiment of MCF7 and PC3 antagomiR-451 cells compared with miR-451 mimic in mice (FIGS. 4A & 4B, respectively). Significant results (P<0.05) are marked with an asterisk.

### Example 4B: In Vivo Model Showing Sensitivity to NAMPT Inhibitor Based on miR-451 Expression

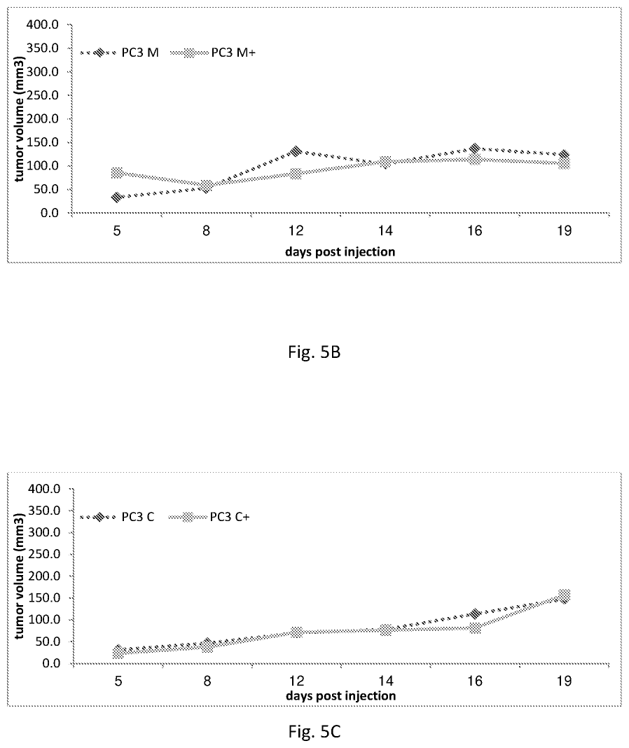

The effect of miR-451 expression levels on efficacy of NAMPT inhibitor was evaluated in an in-vivo xenograft mouse model as in Example 4A. The sensitivity of each prostate cancer cell line (miR-451, antagomiR-451 and scramble miR control) to FK866 treatment was examined in vivo. Twenty mice were injected with each cell line and were divided into two groups: 10 mice were treated with FK866 (15 mg/kg) once a day for 5 days repeated weekly and 10 served as controls, in which no NAMPT inhibitor was injected. The volume of the tumors was measured daily and mice were treated with FK866 from the day all tumors were clinically evident. Treatment with the drug started from day 13 and within 5 days of treatment a change in tumor size was observed between the antagomiR-451 group treated with FK866 and the antagomiR-451 receiving no treatment (FIG. 5A). While mice injected with antagomiR-451 cells showed a major change in tumor growth between the non-treated and the FK866 treated group, a moderate to almost no change in tumor growth between the non-treated and the FK866 treated group was shown in the mice injected with miR-451 mimic cells and control group (FIGS. 5B and 5C).

This model indicates that malignant cells expressing low levels of miR-451 (as evidence by the anagomiR-451 cell line) were more sensitive to FK866 treatment than miR-451 mimic cells, expressing high levels of miR-451 in the mouse xenograft model. Accordingly, the inventors suggest identifying patients in which breast, prostate and colon cancer tumors express low levels of miR-451 and those patients should be preferably treated with a NAMPT inhibitor. Similarly, the inventors suggest identifying patients in which breast, prostate and colon cancer tumors express high levels of miR-451 and those patients should be preferably treated with treatments that do not include a NAMPT inhibitor.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for prognosis of a treatment modality in a patient afflicted with a solid cancerous tumor, the method comprising:
obtaining a biological sample from the patient;
determining a level of expression of miR-451 in the biological sample; and
if the level of expression of miR-451 is below a predetermined level, correlating the level of expression of miR-451 with a positive prognosis of a treatment modality that affects one or more metabolic pathways associated with miR-451 expression.

2. The method according to claim 1, wherein the metabolic pathway associated with miR-451 expression is a NAMPT pathway.

3. The method according to claim 1, wherein the treatment modality that affects one or more metabolic pathway associated with miR-451 expression is a combination therapy.

4. The method according to claim 3, wherein the combination therapy comprises inhibition of a NAMPT pathway combined with one or more of: inhibition of a PARP pathway, radiation, enhancement of DNA/RNA alkylation, inhibition of thymidylate synthase, administration of a nucleotide antimetabolite, inducing TNF-related apoptosis, inhibition of histone deacetylase, administration of an anti-CD20 antibody, inhibition of a proteasome, or inhibition of lactate dehydrogenase A.

5. The method according to claim 1, wherein the solid tumor is selected from the group consisting of: prostate cancer, breast cancer, and colon cancer.

6. A composition for use in treatment of a solid tumor in a patient in need thereof, comprising a therapeutically effective amount of an inhibitor of a NAMPT pathway, wherein the level of expression of miR-451 in the patient is below a predetermined level.

7. The composition according to claim 6 wherein the inhibition of a NAMPT pathway is further combined with one or more of: inhibition of a PARP pathway, radiation, a DNA/RNA alkylation induction, thymidylate synthase inhibition, administration of a nucleotide antimetabolite, TNF-related apoptosis induction, histone deacetylase inhibition, administration of an anti-CD20 antibody, proteasome inhibition, or a lactate dehydrogenase A inhibition.

8. The composition according to claim 6 wherein the level of expression of miR-451 in the patient is determined from a biological sample derived from a tumor from the patient.

9. The composition according to claim 6 wherein the inhibitor of the NAMPT pathway is FK866.

10. The composition according to claim 6, wherein the solid tumor is selected from prostate cancer, breast cancer, or colon cancer.

11. A method for treatment of a solid cancerous tumor in a patient, comprising:
detecting a level of expression of miR-451 in a biological sample from the patient;
assessing if a treatment modality that affects one or more metabolic pathways associated with miR-451 expression will result in a positive prognosis for the patient, and
treating the patient with a treatment modality that affects a metabolic pathway associated with miR-451 expression.

12. The method of claim 11, wherein the treatment modality inhibits the metabolic pathway associated with miR-451 expression.

13. The method of claim 12, wherein the treatment modality induces the metabolic pathway associated with miR-451 expression.


SEQUENCE LISTING (3 sequences)

SEQ ID NO: 1 - RNA, 22 nt, Artificial Sequence
  synthetic polynucleotide
aaaccguuaccauuacugaguu

SEQ ID NO: 2 - RNA, 25 nt, Artificial Sequence
  synthetic polynucleotide
cuuccucucuuucucucccuuguga

SEQ ID NO: 3 - DNA, 18 bp, Artificial Sequence
  synthetic polynucleotide
aatgatctgtaacggttt